(19) United States
(12) Patent Application Publication
(10) Pub. No.: US 2010/0261291 A1
(43) Pub. Date: Oct. 14, 2010

(54) Nucleic acids specifically binding bioactive ghrelin

(75) Inventors: SVEN KLUSSMANN, BERLIN (DE); STEFFEN HELMLING, BOSTON, MA (US); DIRK EULBERG, BERLIN (DE); CHRISTIAN MAASCH, BERLIN (DE); KLAUS BUCHNER, BERLIN (DE)

Correspondence Address: MDIP LLC, POST OFFICE BOX 2630, MONTGOMERY VILLAGE, MD, 20886-2630, US

(73) Assignee: NOXXON Pharma AG, Berlin (DE)

(21) Appl. No.: 12/711,415

(22) Filed: Feb. 24, 2010

Related U.S. Application Data
(63) Continuation of application No. 10/578,938, filed on Jul. 25, 2006.

(30) Foreign Application Priority Data
Nov. 10, 2003 (EP) 03025743.0

(51) Int. Cl. G01N 33/53; C07H 21/04

(52) U.S. Cl. 436/501; 536/23.1

(57) ABSTRACT

The present invention is related to a nucleic acid specifically binding bioactive ghrelin, more preferably n-octanoyl ghrelin, and its use for the diagnosis of ghrelin mediated diseases and disorders.

The present invention is related to nucleic acids which bind to a bioactive ghrelin, and the use of such nucleic acid for the binding and detection of bioactive ghrelin.

Ghrelin was identified as the natural ligand of the growth hormone secretagogue receptor 1a (GHSR1a). The receptor is most abundant in the pituitary gland and in hypothalamic parts of the brain, but can also be detected in other tissues at low concentrations. Since the late 70ies synthetic peptides and other compounds, named secretagogues had been shown to stimulate the release of growth hormone. However, the natural ligand responsible for the release of growth hormone remained unknown until the discovery of ghrelin in 1999. Ghrelin is a highly basic 28 amino acid peptide hormone with an octanoyl acid side chain at the third amino acid of its N-terminus (serine 3). This unusual modification is required for the interaction at the GHS-receptor and its activity. However, in biological samples a mixture of both, the octanoyl ghrelin which is a form of a bioactive ghrelin and the unmodified or des-octanoyl ghrelin which is present. The amino-acid sequence of the purified rat ghrelin was determined by a protein sequencer to be GSSFLSPEHQKAQQRKESKKPPAKLQPR (SEQ. ID. No. 19). The corresponding human sequence deviates in two positions only, carrying the same n-octanoyl-side chain at the amino acid position serine 3 (GSSFLSPEHQRVQQRKESKKPPAKLQPR (SEQ. ID. No. 16).

Beside the naturally occurring n-octanoyl residue, unsaturated or branched octanoyl groups, and longer aliphatic chains introduced at position 3 of ghrelin mediate receptor recognition as well. The receptor interaction domain is located at the very N-terminus of ghrelin; deletion studies indicate, that ghrelin (1-10) [GSSFLSPEHQ, SEQ. ID No. 17] and even the minimal motif of amino acids 1-5 (ghrelin (1-5) [GSSFL, SEQ. ID. No. 18]) are sufficient for stimulation of GHSR1a, but in both cases, a strong requirement for peptide modification with the n-octanoyl residue is observed.

Ghrelin has been shown to mediate physiological functions pertinent to an anabolic state. While it directly stimulates the release of growth hormone (GH) from the pituitary gland, experiments in rodents also showed ghrelin to induce feeding in a GH-independent fashion by acting upon hypothalamic neurons. Interestingly, the primary site of ghrelin production is in oxyntic glands in the stomach, suggesting that it serves as a hormonal link between stomach, pituitary gland and hypothalamus. The observation that ghrelin administration in rats resulted in weight gain as a consequence of changes in energy intake and/or fuel utilization is in support of such a role. Moreover, systemic ghrelin administration in humans cause sensations of hunger in the test subjects and induce overeating. Based on these findings ghrelin is thought to have a crucial role in the regulation of appetite and body weight, serving as an acute as well as a chronic signal of an underfed state. Additional support for this hypothesis comes from observations that ghrelin levels as well as appetite are reduced in individuals following gastric bypass, contributing at least in part to the efficiency of the procedure in effecting weight loss. Clinical data from patients with Prader-Willi syndrome also suggest that the hyperphagia and obesity associated with the disease are a consequence of tremendous hyperghrelinemia. Moreover, ghrelin was found to induce hyperglycemia and inhibition of insulin release, indicating an involvement in glucose metabolism. Beside these functions in energy metabolism, ghrelin has also been implicated in a number of other processes. It was found to be expressed in a number of neuroendocrine tumors and to stimulate, besides GH release from the pituitary, the release of ACTH, PRL, and cortisol. Single injections of ghrelin into healthy individuals were found to increase cardiac output and decrease blood pressure. Thus, ghrelin action appears to be involved in a variety of different tasks. For background information may be taken from M. Kojima, H. Hosoda, Y. Date, M. Nakazato, H. Matsu, K. Kangawa, “Ghrelin is a growth-hormone-releasing acylated peptide from stomach”, Nature 402:656-60, 1999; M. Tschöp, D. L. Smiley, M. L. Heiman, “Ghrelin induces adiposity in rodents”, Nature 407:908-13, 2000; A. M. Wren et al., “Ghrelin enhances appetite and increases food intake in humans”, Journal of Clinical Endocrinology Metabolism 86:5992-6, 2001; M. Nakazato et al., “A role for ghrelin in the central regulation of feeding”, Nature 409: 194-8, 2001; N. Nagaya, et al., Am J Physiol Regul Integr Comp Physiol. 2001 May; 280 (5):R1483-7; Hemodynamic and hormonal effects of human ghrelin in healthy volunteers; Volante M, et al., J Clin Endocrinol Metab. 2002 March; 87(3):1300-8. Expression of ghrelin and of the GH secretagogue receptor by pancreatic islet cells and related endocrine tumors; Jeffery P L, et al., J. Endocrinol. 2002 March; 172 (3):R7-11 Expression and action of the growth hormone releasing peptide ghrelin and its receptor in prostate cancer cell lines; Egido E M, et al., Eur J. Endocrinol. 2002 February; 146(2):241-4 Inhibitory effect of ghrelin on insulin and pancreatic somatostatin secretion; Broglio F, et al., J Clin Endocrinol Metab. 2001 October; 86(10):5083-6, Ghrelin, a natural GH secretagogue produced by the stomach, induces hyperglycemia and reduces insulin secretion in humans; Bednarek M A, et al., J Med. Chem. 2000 October; 43:4370-6 Structure-function studies on the new growth hormone-releasing peptide, ghrelin: minimal sequence of ghrelin necessary for activation of growth hormone secretagogue receptor 1a.

The problem underlying the present invention is to provide means for the binding of bioactive ghrelin and more particularly to provide a method for the treatment of diseases and disorders mediated by bioactive ghrelin as well as methods for the specific detection of bioactive ghrelin.

According to the present invention the problem is solved by the subject matter of the independent claims which are attached hereto. Preferred embodiments result from the dependent claims.

Human ghrelin is a basic peptide having the amino acid sequence according to SEQ. ID. No. 16, and is modified with a fatty acid side chain. In consideration of the high degree of peptide sequence homology between different species, the term ghrelin used herein refers to any ghrelin including, but not limited to, mammalian ghrelin. Preferably, the mammalian ghrelin is selected from the group comprising mice, rat, rabbit, hamster and human ghrelin. Most preferably the ghrelin is human ghrelin.

The calculated pI of ghrelin is 11.09. Despite of this very basic over-all pI of ghrelin, the receptor binding motif GSSFL [ghrelin (1-5)] is a rather acidic domain, with a calculated pI of 5.5. The present invention is based on the surprising finding, that a nucleic acid can be selected with full-length ghrelin, that specifically recognizes the acidic receptor binding domain, but not the basic central and carboxy-terminal domain of the peptide. This is surprising in regard of electrostatic effects of both the charges of target molecule, i.e. ghrelin, and the charges of the nucleic acid. The binding of negatively charged nucleic acids to a basic domain of a target molecule should be much more advantageous compared to the binding of a nucleic acid to an acidic domain of a target molecule. Thus it has to be pointed out that the one skilled in the art had no reasonable expectation of success to select a nucleic acid ligand that is not binding to the basic part of ghrelin but is binding to the acidic domain of the target molecule.

Beside the amino-terminal receptor binding motif, biologically active ghrelin which is also referred to herein as bioactive ghrelin, is characterized by its acylation with a n-octanoly group at amino acid serine 3. The nucleic acid ligand of the amino-terminal motif GSSFL disclosed herein allows the discrimination of the biologically active from the bio-inactive or non-bioactive form of ghrelin. This is surprising, since binding is strictly dependent on the presence of two moieties, the octanoyl group and the peptide: binding of the nucleic acid to octanoyl-ghrelin is specific in the presence of a 1000-fold excess of desoctanoyl-ghrelin, more preferable in the presence in a 100-fold excess of desoctanoyl-ghrelin, and most preferable in the presence of a 10-fold excess of desoctanoyl-ghrelin. Furthermore, the binding characteristics are also specific for the peptide moiety, given the fact, that the enantiomeric octanoyl-ghrelin is not recognized by the nucleic acid; the octanoyl-group is not sufficient for binding.

As used in preferred embodiments herein, a bioactive ghrelin is a ghrelin which exhibits in a preferred embodiment essentially all of the characteristics of the naturally occurring ghrelin. Particularly, a bioactive ghrelin as used herein in preferred embodiments is any ghrelin and ghrelin derivative which is responsible for or can trigger the release of growth hormone, more preferably via an interaction with the GHS receptor. In contrast to this in preferred embodiments a non-bioactive ghrelin is a ghrelin which is different from bioactive ghrelin, more preferably does not trigger the release of growth hormone, more preferably via an interaction with the GHS receptor.

The features of the nucleic acid according to the present invention as described herein can be realised in any aspect of the present invention where the nucleic acid is used, either alone or in any combination.

The nucleic acid according to the present invention also comprises nucleic acids which are essentially homologous to the particular sequences disclosed herein. The term substantially homologous shall be understood such as the homology is at least 75%, preferably 85%, more preferably 90% and most preferably more that 95%, 96%, 97%, 98% or 99%.

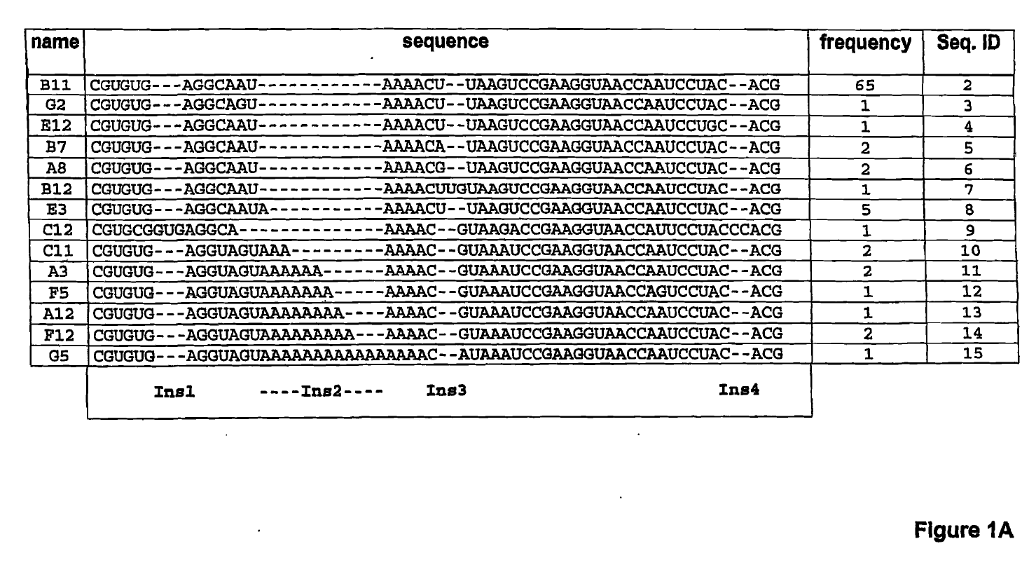

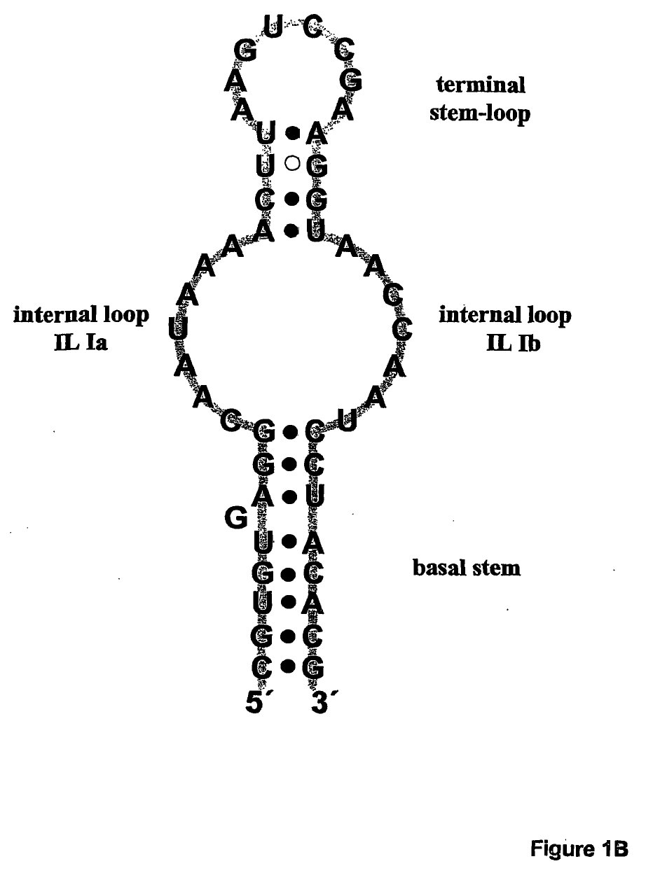

The nucleic acid according to the present invention also comprises in an embodiment a nucleic acid which is derived from the particular sequences disclosed herein. The term ‘derived’ shall be understood such as on the basis of SEQ. ID No. 1 the insertion loci Ins1 to Ins4 shown in FIG. 1A can be represented by any sequence of a length of a maximum of 30 nucleotides, preferable by any sequence of a maximum of 20 nucleotides, more preferable by any sequence of a maximum of 10 nucleotides, and most preferable by any sequence of 0-3 nucleotides for Ins1, 0-14 nucleotides for Ins2, 1-3 nucleotides for Ins 3, and 0-2 nucleotides for Ins4. The internal loop IL Ia, represented by Ins2, is considered to be the most important site of modification.

The nucleic acid according to the present invention can also be represented in a preferred embodiment by the following generic formula

(SEQ. ID. No. 1)

CGUGYGN₍₀₋₃₎AGGYAN₍₀₋₁₄₎AAAACN₍₁₋₃₎UAARWCCGAAGGUAA
CCAWUCCUACN₍₀₋₂₎ACG

whereby Y stands for U or C, R stands for A or G, W stands for U or A. In connection therewith it is to be notes that any of the indices represent any integer starting from the first figure specified to the last figure specified and any integer therebetween. Accordingly, e.g. 0-3 represent 0, 1, 2 and 3.

Thus, the consensus sequence SEQ. ID. No. 1 contains four regions, where insertions of variable length are observed in various embodiments. These regions are called insertion loci, and are labelled Ins1 to Ins 4. According to L-NOX-B11, listed as SEQ. ID. No. 2 in FIG. 1A, Ins1 is located at between nucleotides 6 and 7, Ins2 is located between nucleotides 13 and 14, Ins3 is located between nucleotides 18 and 20, and Ins4 is located between nucleotides 44 and 45. The length of the respective insertion loci, observed in the depicted clones, is given in SEQ. ID. No. 1. and the above specified generic formula.

The nucleic acid according to the present invention also comprises in an embodiment a nucleic acid which is structurally homologue to the particular sequences disclosed herein, preferably to the extent that said parts are involved in binding to octanoyl-ghrelin and discriminating des-octanoyl ghrelin. Structural homology as used in connection with preferred embodiments of the present invention shall be understood such as the sequences fold into a characteristic secondary structure model comprising a basal stem, and internal loop, and a terminal stem-loop as depicted in FIG. 1B, preferable folding into said structure, where in stem regions compensatory base exchanges occur, and preferable folding into said structure, where in single-stranded stretches substitutions, deletions and/or insertions occur, and most preferable folding into said structure corresponding to FIG. 1B in size and to SEQ. ID. 1 in sequence.

The term inventive nucleic acid or nucleic acid according to the present invention shall also comprise those nucleic acids comprising part of the nucleic acids sequences disclosed herein, preferably to the extent that said parts are involved in the binding to ghrelin, and discriminating bioactive ghrelin from non-bioactive ghrelin, i.e. in particular octanoyl-ghrelin from des-octanoyl-ghrelin. Such a nucleic acid may be derived from the ones disclosed herein, e.g., by truncation. Truncation may be related to either or both of the ends of the nucleic acids as disclosed herein. Also, truncation may be related to the inner sequence of nucleotides, i.e. it may be related to the nucleotide(s) between the 5′ and the 3′ terminal nucleotide, respectively. Moreover, truncation shall comprise the deletion of as little as a single nucleotide from the sequence of the nucleic acids disclosed herein. Truncation may also be related to more than one stretch of the inventive nucleic acid(s), whereby the stretch can be as little as one nucleotide long.

The nucleic acids according to the present invention may be either D-nucleic acids or L-nucleic acids. Preferably, the inventive nucleic acids are L-nucleic acids. In addition it is possible that one or several parts of the nucleic acid are present as D-nucleic acids or at least one or several parts of the nucleic acids are L-nucleic acids. The term “part” of the nucleic acids shall mean as little as one nucleotide. Such nucleic acids are generally referred to herein as D- and L-nucleic acids, respectively.

The term inventive nucleic acid or nucleic acid according to the present invention shall also comprise those nucleic acids that comprise the nucleic acids sequences disclosed herein and other sequences attached thereto, preferably to the extent that said parts or nucleic acids are involved in the binding to octanoyl-ghrelin and discriminating desoctanoyl-ghrelin. The extension i.e. additional sequences attached to the specific nucleic acid sequences disclosed herein may be such, that the sequence is elongated either at the 5′-terminus or the 3′-terminus or both, and it may comprise as much as 100 nucleotides for either side, preferably as much as 50 nucleotides for either side, more preferably as much as 20 nucleotides on either side, and most preferably the complete or partial 5′-flank sequence which is disclosed herein as SEQ. ID. No. 20, and/or the complete or partial 3′-flank sequence which is disclosed herein as SEQ. ID. No. 21. As used herein, the term partially means in a preferred embodiment of the present invention a single nucleotide of the respective sequence or a sequence of two or more nucleotides of such sequence which are adjacent to each other in the sequence to which it is referred to, more particularly to the flank sequences according to any of SEQ. ID. No. 20 and 21.

It is also within the present invention that the nucleic acids according to the present invention are part of a longer nucleic acid whereby this longer nucleic acid comprises several parts whereby at least one part is a nucleic acid, or a part thereof, according to the present invention. The other part of these longer nucleic acids can be either a D-nucleic acid or L-nucleic acid. Any combination may be used in connection with the present invention. These other part(s) of the longer nucleic acid can exhibit a function which is different from binding. One possible function is to allow interaction with other molecules such as, e.g., for immobilization, cross-linking, detection or amplification.

L-nucleic acids as used herein are nucleic acids consisting of L-nucleotides, preferably consisting completely of L-nucleotides.

D-nucleic acids as used herein are nucleic acids consisting of D-nucleotides, preferably consisting completely of D-nucleotides.

Irrespective of whether the inventive nucleic acid consists of D-nucleotides, L-nucleotides or a combination of both with the combination being e.g. a random combination or a defined sequence of stretches consisting of at least one L-nucleotide and at least one D-nucleic acid, the nucleic acid may consist of desoxyribonucleotide(s), ribonucleotide(s) or combinations thereof.

Designing the inventive nucleic acids as L-nucleic acid is advantageous for several reasons. L-nucleic acids are enantiomers of naturally occurring nucleic acids. D-nucleic acids, however, are not very stable in aqueous solutions and particularly in biological systems or biological samples due to the widespread presence of nucleases. Naturally occurring nucleases, particularly nucleases from animal cells are not capable of degrading L-nucleic acids. Because of this the biological half-life of the L-nucleic acid is significantly increased in such a system, including the animal and human body. Due to the lacking degradability of L-nucleic acid no nuclease degradation products are generated and thus no side effects arising therefrom observed. This aspect delimits the L-nucleic acid of factually all other compound which are used in the therapy of diseases and/or disorders involving the presence of ghrelin.

It is also within the present invention that the inventive nucleic acids, regardless whether they are present as D-nucleic acids, L-nucleic acids or D,L-nucleic acids or whether they are DNA or RNA, may be present single stranded or double stranded nucleic acids. Typically, the inventive nucleic acids are single stranded nucleic acids which exhibit defined secondary structures due to the primary sequence and may thus also form tertiary structures. The inventive nucleic acids, however, may also be double stranded in the meaning that two strands which are complementary to each other are hybridised to each other. This confers stability to the nucleic acid which will be advantageous if the nucleic acid is present in the naturally occurring D-form rather than the L-form.

The inventive nucleic acids may be modified. Such modifications may be related to the single nucleotide of the nucleic acid and are well known in the art. Examples for such modification are described in, among others, Kusser, W. (2000) J Biotechnol, 74: 27-38; Aurup, H. et al. (1994) Nucleic Acids Res, 22, 20-4; Cummins, L. L. et al, (1995) Nucleic Acids Res, 23, 2019-24; Eaton, B. E. et al. (1995) Chem Biol, 2, 633-8; Green, L. S. et al., (1995) Chem Biol, 2, 683-95; Kawasaki, A. M. et al., (1993) J Med Chem, 36, 831-41; Lesnik, E. A. et al., (1993) Biochemistry, 32, 7832-8; Miller, L. E. et al., (1993) J Physiol, 469, 213-43.

The nucleic acids according to the present invention may be a multipartite nucleic acid. A multipartite nucleic acid as used herein, is a nucleic acid which consists of at least two nucleic acid strands. These at least two nucleic acid strands form a functional unit whereby the functional unit is a ligand to a target molecule. The at least two nucleic acid strands may be derived from any of the inventive nucleic acids by either cleaving the nucleic acid to generate two strands or by synthesising one nucleic acid corresponding to a first part of the inventive, i.e. overall nucleic acid and another nucleic acid corresponding to the second part of the overall nucleic acid. It is to be acknowledged that both the cleavage and the synthesis may be applied to generate a multipartite nucleic acid where there are more than two strands as exemplified above. In other words, the at least two nucleic acid strands are typically different from two strands being complementary and hybridising to each other although a certain extent of complementarity between the various nucleic acid parts may exist.

A possibility to determine the binding constant is the use of the so called biacore device, which is also known to the one skilled in the art. Affinity as used herein was also measured by the use of “bead assays” as described in example 5. An appropriate measure in order to express the intensity of the binding between the nucleic acid according to the target which is in the present case ghrelin, is the so-called Kd value which as such as well the method for its determination are known to the one skilled in the art.

The nucleic acids according to the present invention are characterized by a certain Kd value. Preferably, the Kd value shown by the nucleic acids according to the present invention is below 1 μM. A Kd value of about 1 μM is said to be characteristic for a non-specific binding of a nucleic acid to a target. As will be acknowledged by the ones in the art, the Kd value of a group of compounds such as the nucleic acids according to the present invention are within a certain range. The above-mentioned Kd of about 1 μM is a preferred upper limit for the Kd value. The preferred lower limit for the Kd of target binding nucleic acids can be about 10 picomolar or higher. It is within the present invention that the Kd values of individual nucleic acids discriminating bioactive ghrelin from non-bioactive ghrelin, i.e. preferably octanoyl-ghrelin from desoctanoyl-ghrelin are with in this range of 10 pM to 1 μM, more preferred within a range of 100 pM to 500 nM, and most preferred within a range of 1 nM to 100 nM.

The nucleic acid molecules according to the present invention may have any length provided that they are still able to bind to the target molecule, and discriminate bioactive ghrelin from non-bioactive ghrelin, i.e. preferably octanoyl-ghrelin from desoctanoyl-ghrelin. It will be acknowledged in the art that there are preferred lengths of the nucleic acids according to the present inventions. Typically, the length is between 15 and 120 nucleotides. It will be acknowledged by the ones skilled in the art that any integer between 15 and 120 is a possible length for the nucleic acids according to the present invention. More preferred ranges for the length of the nucleic acids according to the present invention are lengths of about 20 to 100 nucleotides, about 20 to 80 nucleotides, about 20 to 60 nucleotides, about 20 to 50 nucleotides and about 30 to 50 nucleotides.

The assays for discrimination of bioactive and bio-inactive ghrelin according to the present invention may be performed using standard techniques as known by persons skilled in the art. In a preferred aspect, the assays may be performed in 96-well plates, where components are immobilized in the reaction vessels as disclosed according to the claims. Optionally, the complexes can be removed from the reaction vessels after complex formation.

In one aspect, the nucleic acid molecule according to the invention is analysed by a second detection means, wherein the said detection means is a molecular beacon. The methodology of molecular beacon is known to persons skilled in the art. In brief, nucleic acids probes which are also referred to as molecular beacons, are a reverse complement to the nucleic acids sample to be detected and hybridise because of this to a part of the nucleic acid sample to be detected. Upon binding to the nucleic acid sample the fluorophoric groups of the molecular beacon are separated which results in a change of the fluorescence signal, preferably a change in intensity. This change correlates with the amount of nucleic acids sample present.

The inventive nucleic acids, which are also referred to herein as the nucleic acids according to the present invention, and/or the antagonists according to the present invention may be used for the generation or manufacture of a medicament. Such medicament contains at least one of the inventive nucleic acids, optionally together with further pharmaceutically active compounds, whereby the inventive nucleic acid preferably acts as pharmaceutically active compound itself. Such medicaments comprise in preferred embodiments at least a pharmaceutically acceptable carrier. Such carrier may be, e.g., water, buffer, starch, sugar, gelatine or any other acceptable carrier substance. Such carriers are generally known to one skilled in the art. Disease and/or disorders and/or diseased conditions for the treatment and/or prevention of which such medicament may be used include, but are not limited to obesity, the regulation of energy balance, appetite and body weight, eating disorders, diabetes, glucose metabolism, tumour, blood pressure and cardiovascular diseases. As will be acknowledged by the ones of the art the inventive nucleic acids may factually be used in any disease where an antagonist to ghrelin can be administered to a patient in need of such antagonist and such antagonist is suitable to eliminate the cause of the disease or the disorder or at least to reduce the effects from the disease or the disorder. Such effect includes, but is not limited to obesity, the regulation of energy balance, appetite and body weight, eating disorders, diabetes, glucose metabolism, tumour treatment, blood pressure and cardiovascular diseases. For the purpose of the present invention regulation of energy balance is regarded as a disease. More particularly, the use is for the treatment of any disease where the regulation of the energy balance is influenced by ghrelin, either directly or indirectly, and whereby reduction of the bioavailability of ghrelin is desired. The same applies to sugar metabolism, blood pressure and appetite and body weight. Further disease which may be treated using the nucleic acids according to the present invention, possibly upon systemic or local application are those which can be selected from the group comprising pituitary tumors, acromegaly, central Cushing's syndrome, adrenal Cushing's syndrome, paraneoplastic Cushing's syndrome, ectopic Cushing's syndrome, adrenal tumor, stress, hypercortisolism, cardiac insufficiency, cardiay infarction, stroke, adrenocortical insufficiency, hypotonia, aortic stenosis, pulmonal hypertonia, constrictive pericarditis, infectious diseases, infectious toxic hypotonia, hypovolemia, and hypronatriemia.

It is to be understood that the nucleic acid as well as the antagonists according to the present invention can be used not only as a medicament or for the manufacture of a medicament, but also for cosmetic purposes, particularly with regard to the involvement of ghrelin in obesity. For the same purpose the nucleic acid as well as the antagonists according to the present invention can be used as a food additive, a means for weight control and/or a means for appetite control. A composition comprising the nucleic acid as well as the antagonists according to the present invention can be used for any of the aforementioned purposes.

The inventive nucleic acid may further be used as starting material for drug design. Basically there are two possible approaches. One approach is the screening of compound libraries whereas such compound libraries are preferably low molecular weight compound libraries. Such libraries are known to the one skilled in the art. Alternatively, the nucleic acid according to the present invention may be used for rational design of drugs.

The rational design of drugs may start from any of the nucleic acid according to the present invention and involves a structure, preferably a three dimensional structure, which is similar to the structure of the inventive nucleic acids or identical to the binding mediating parts of the structure of the inventive nucleic acids. In any case such structure still shows the same or a similar binding characteristic as the inventive nucleic acids. In either a further step or as an alternative step in the rational design of drugs the preferably three dimensional structure of those parts of the nucleic acids binding to the neurotransmitter are mimicked by chemical groups which are different from nucleotides and nucleic acids. By this mimicry a compound different from the nucleic acids can be designed. Such compound is preferably a small molecule or a peptide.

In case of screening of compound libraries, such as by using a competitive assay which are known to the one skilled in the arts, appropriate ghrelin analogues, ghrelin agonists or ghrelin antagonists may be found. Such competitive assays may be set up as follows. The inventive nucleic acid, preferably a spiegelmer which is a target binding L-nucleic acid, is coupled to a solid phase. In order to identify ghrelin analogues labelled ghrelin may be added to the assay. A potential analogue would compete with the ghrelin molecules binding to the spiegelmer which would go along with a decrease in the signal obtained by the respective label. Screening for agonists or antagonists may involve the use of a cell culture assay as known to the ones skilled in the art.

The kit according to the present invention may comprise at least one or several of the inventive nucleic acids. Additionally, the kit may comprise at least one or several positive or negative controls. A positive control may, for example, be ghrelin, particularly the one against which the inventive nucleic acid is selected or to which it binds, preferably, in liquid form. A negative control may, e.g., be a peptide which is defined in terms of biophysical properties similar to ghrelin, but which is not recognized by the inventive nucleic acids. Furthermore, said kit may comprise one or several buffers. The various ingredients may be contained in the kit in dried or lyophilised form or solved in a liquid. The kit may comprise one or several containers which in turn may contain one or several ingredients of the kit.

It is to be understood that any of the sequences disclosed in the examples and the figures, respectively, is disclosed as such and any such sequence can be used in any aspect and embodiment of the present invention.

The present invention is further illustrated by the figures, examples and the sequence listing from which further features, embodiments and advantages may be taken, wherein

FIG. 1A shows the members of the L-NOX-B11 group, their name, the frequency by which they were selected, and their truncated sequence, which may alternatively be extended by the 5′-flank 5′-GGAGCUCAGACUUCACU-3′ (SEQ. ID. No. 20) and the 3′-flank 5′-UACCACUGUCGGUUCCAC-3′ (SEQ. ID. No. 21), and indicates the insertion loci Ins1 to Ins4;

FIG. 1B shows the secondary structure model of the truncated clone L-NOX-B11 and indicates the regions of the basal stem, the 5′- and the 3′-part of the internal loop (IL Ia, ILIb), and the terminal stem-loop;

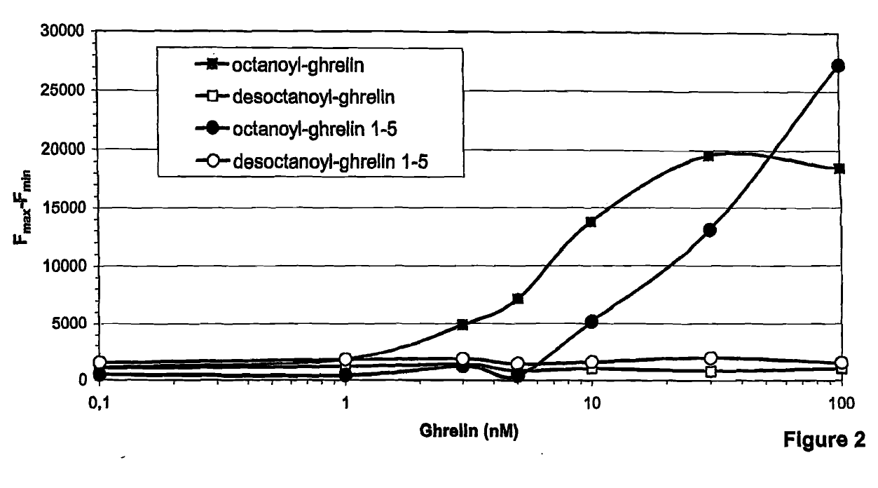

FIG. 2 shows the dose-dependent calcium release mediated by octanoyl- or desoctanoyl-ghrelin in the full-length or the truncated form in a cellular assay using CHO cells expressing human ghrelin receptor (dose-response titration);

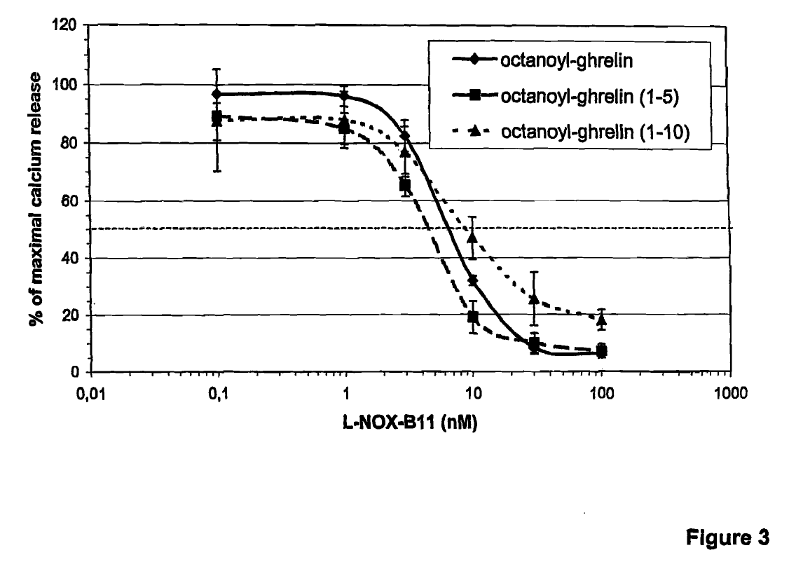

FIG. 3 shows the inhibition of calcium release mediated by full-length and truncated octanoyl-ghrelin by the Spiegelmer L-NOX-B11 (inhibition curve);

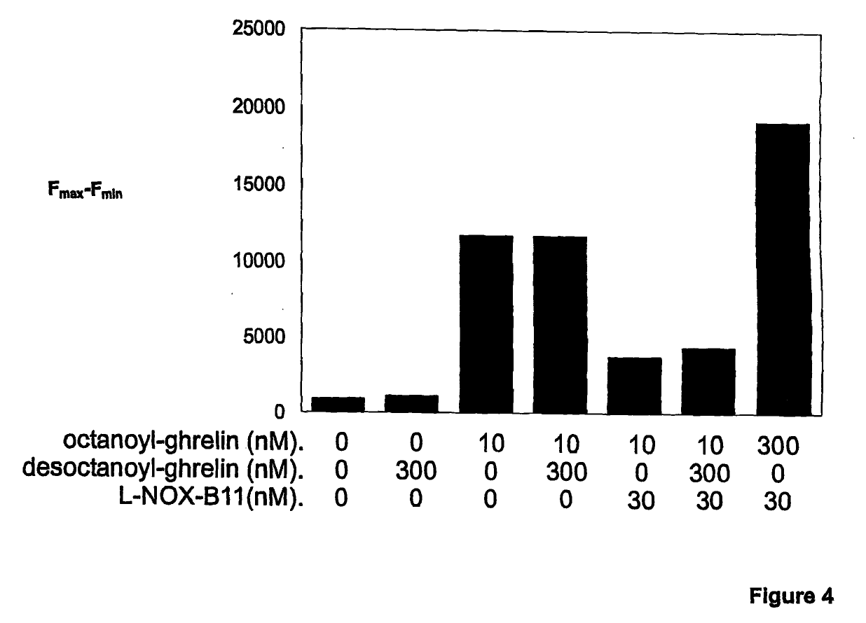

FIG. 4 shows the results of a cellular competition assay with octanoyl-ghrelin, desoctanoyl-ghrelin, and L-NOX-B11, with combinations and concentrations of the components summarized below the bars;

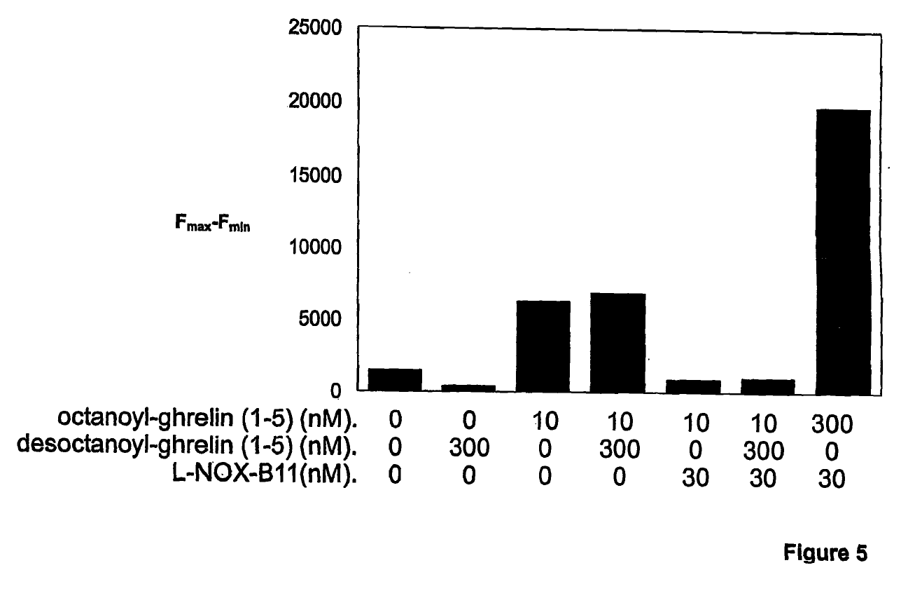

FIG. 5 shows the results of a cellular competition assay with octanoyl-ghrelin (1-5), desoctanoyl-ghrelin (1-5), and L-NOX-B11, with combinations and concentrations of the components summarized below the bars;

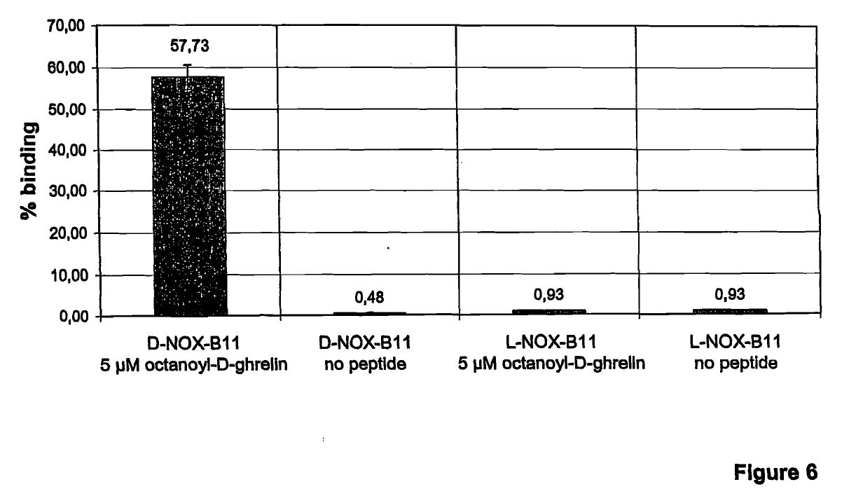

FIG. 6 shows results of an in vitro binding assay, analysing the binding of radio-labelled D-NOX-B11 and L-NOX-B11 to biotinylated D-octanoyl-ghrelin.

The following table links the SEQ. ID. Numbers to the various clones and identifiers, respectively, described herein. Nucleic acids sequences, if not indicated in a contrary way, are represented as the (+) strands and built by 2′OH-ribonucleotides.

TABLE

| Sequence | Sequence type | SEQ. ID. No |
|---|---|---|
| consensus sequence | nucleic acid | 1 |
| L-NOX-B11 group | | |
| L-NOX-B11 | nucleic acid | 2 |
| L-NOX-G2 | nucleic acid | 3 |
| L-NOX-E12 | nucleic acid | 4 |
| L-NOX-B7 | nucleic acid | 5 |
| L-NOX-A8 | nucleic acid | 6 |
| L-NOX-B12 | nucleic acid | 7 |
| L-NOX-E3 | nucleic acid | 8 |
| L-NOX-C12 | nucleic acid | 9 |
| L-NOX-C11 | nucleic acid | 10 |
| L-NOX-A3 | nucleic acid | 11 |
| L-NOX-F5 | nucleic acid | 12 |
| L-NOX-A12 | nucleic acid | 13 |
| L-NOX-F12 | nucleic acid | 14 |
| L-NOX-G5 | nucleic acid | 15 |
| ghrelin (human) | peptide | 16 |
| human ghrelin (1-10) | peptide | 17 |
| human ghrelin (1-5) | peptide | 18 |
| ghrelin (rat) | peptide | 19 |
| 5′-flank sequence | nucleic acid | 20 |
| 3′-flank sequence | nucleic acid | 21 |

### EXAMPLE 1

### Ghrelin-Binding Nucleic Acid Ligands

In the European Patent Application EP 020 23 627.8 and the International Patent Application PCT/EP03/08542 the generation of ghrelin-binding nucleic acid ligands is described. One group of such nucleic acid ligands, obtained in the selection process is shown in FIG. 1A. The clone L-NOX-B11 is the most abundant sequence in this group, and—like all the other members of the group—is functional in a long and a truncated version (L-NOX-B11 [86] and L-NOX-B11 [47]). For elongation of the truncated clones, the 5′-flank and the 3′-flank sequences may be added to the core sequence shown.

| | | |
|---|---|---|
| 5′-flank | 5′-GGAGCUCAGACUUCACU-3′ | SEQ. ID. No. 20 |
| 3′-flank | 5′-UACCACUGUCGGUUCCAC-3′ | SEQ. ID. No. 21 |

In FIG. 1A the truncated versions only are summarized, and in this patent application, results concerning these truncated clone are presented only. However, characteristics of L-NOX-B11 [47] disclosed herein do also concern all elongated versions of all truncated sequences.

The individual clones in the L-NOX-B11-group are highly conserved and show long stretches of sequence identity. The following consensus sequence can be gained from the clones shown in FIG. 1A:

(SEQ. ID. No. 1)

CGUGYGN₍₀₋₃₎AGGYAN₍₀₋₁₄₎AAAACN₍₁₋₃₎UAARWCCGAAGGUAA
CCAWUCCUACN₍₀₋₂₎ACG

where Y stands for U or C, R stands for A or G, W stands for U or A.

As can be seen, nucleotide substitution are found only in a few positions. Furthermore, there are 4 defined regions, where sequence insertion occurs; these insertion loci are labelled Ins1 to Ins4 and correspond to the letters ‘N_{(x-y)}’ in SEQ. ID. No. 1. At these positions any nucleotide in any number, preferable in a number given in the brackets in SEQ. ID. No. 1, may be inserted. In the insertion locus 2, the preferred nucleotide inserted is an adenosin residue.

The sequence of L-NOX-B11 folds into a characteristic secondary structure shown in FIG. 1B, comprising a basal stem, an internal loop, and a terminal stem-loop structure. A detailed analysis of all sequences within the group shows, that the insertion loci of the sequence mainly fall into the region of the internal loop (Ins2). The terminal stem-loop as well as the basal stem are always identical and seem to be highly characteristic for this family of ghrelin-binding molecules and their specific features. It need be mentioned, that several sequence substitutions, obvious for a person skilled in the art, that do not or only slightly disrupt the secondary structure given in FIG. 1B, can be done, without loss of the specific function of the nucleic acid, namely in discriminating bioactive ghrelin from the bio-inactive one. In several selections disclosed in the European Patent Application EP 020 23 627.8 and the International Patent Application PCT/EP03/08542, these kind of modified sequences were found. Features described for L-NOX-B11 can be transferred to those sequences, that are sufficiently conserved regarding sequence and structure.

### EXAMPLE 2

### Method to Analyse the Ghrelin-Induced Calcium-Release

Functional characterization of ghrelin-binding Spiegelmers is performed in a cellular assay system monitoring the interaction of ghrelin and the human growth hormone secretagogue receptor (GHS-R). The intracellular calcium release resulting from receptor-ligand interaction is visualized by means of a fluorescent calcium indicator.

Stable transfected CHO-cells expressing the human ghrelin receptor (GHS-R1a) (obtained from Euroscreen, Gosselies, Belgium) are seeded with 5−7×10⁴ cells per well in a black 96 well-plate with clear bottom (Greiner) and cultivated overnight at 37° C. and 5% CO₂ in UltraCHO medium (Cambrex) which contained in addition 100 units/ml penicillin, 100 μg/ml streptomycin, 400 μg/ml geneticin and 2.5 μg/ml fungizone.

Before loading with the calcium indicator dye fluo-4, cells are washed once with 200 μl CHO-U+ (5 mM probenecid, 20 mM HEPES in UltraCHO medium). Then 50 μl of the indicator dye solution (10 μM fluo-4 (Molecular Probes), 0.08% pluronic 127 (Molecular Probes) in CHO-U+) are added and the cells are incubated for 60 min at 37° C. Thereafter cells are washed three times with 180 μl CHO-U+. Finally 90 W CHO-U+ are added per well.

In the stimulation assay, full-length or truncated versions of human or rat L-ghrelin, either in the octanoyl- or desoctanoyl-form, are used as indicated [L-ghrelin and desoctanoyl-L-ghrelin were obtained from Bachem (Basel, Switzerland), and L-ghrelin (1-5), L-ghrelin (1-10), and desoctaboyl-L-ghrelin (1-5) were from Phoenix Pharmaceuticals (Belmont, Calif.)].

The respective peptides are incubated in CHO-U+ for 15 to 60 min at room temperature in a 0.2 ml low profile 96-tube plate. In these stimulation solutions, the peptide is 10-fold concentrated compared to the assay. For detection of calcium release, the stimulation solution is added to the cells (10 μl/well), and the change of the fluorescence signal is monitored. Measurement of fluorescence signals is done at an excitation wavelength of 485 nm and an emission wavelength of 520 nm in a Fluostar Optima multidetection plate reader (BMG).

For parallel measurement of several samples, wells of one (perpendicular) row of a 96 well plate are recorded together. First three readings with a time lag of 4 sec are done for determination of the base line. Then the recording is interrupted and the plate is moved out of the instrument. Using a multi-channel pipette, 10 μl of the stimulation solution is added to the wells, then the plate is moved into the instrument again and the measurement is continued. In total 20 recordings with time intervals of 4 sec are performed.

For each well the difference between maximal fluorescence and base line value (Fₘₐₓ-Fₘᵢₙ) is determined and plotted against ghrelin concentrations. In FIG. 2, the dose response curves of human octanoyl- and desoctanoyl-ghrelin (full-length and truncated peptide) are shown. It turns out, that both, the full-length and the truncated octanoyl-ghrelin induce calcium release, however, to different extends: full length octanoyl-ghrelin shows maximal activity at a concentration of 30 nM, while octanoyl-ghrelin 1-5 only stimulates at higher peptide concentrations and does not reach maximal signal intensity in the concentration range observed. The desoctanoyl-forms of both peptides do not stimulate the human ghrelin receptor at any concentration analysed in the assay. This experiments confirms, that the five N-terminal amino acids of ghrelin are sufficient for stimulation of the human ghrelin receptor, and that the octanoyl-group is essential for the biologic activity of ghrelin.

### EXAMPLE 3

### Inhibition of Ghrelin-Induced Calcium-Release by Ghrelin-Binding Spiegelmers

Inhibition of ghrelin-induced calcium release was measured using the cellular assay described in Example 2. As a modification of the method, the stimulation solutions in the inhibition assay were supplemented with variable amounts of the Spiegelmer L-NOX-B11. As a control, samples with peptide only (maximal calcium release) and samples without peptide (minimal calcium release) were analysed. After incubation for 15-60 minutes at room temperature, 10 μl of the stimulation solutions were added to the cells, resulting in a peptide final concentration of 5 nM. Usually Spiegelmer final concentrations of 0.1 nM, 1 nM, 3 nM, 10 nM, 30 nM, and 100 nM were chosen.

For each well the difference between maximal fluorescence and base line value (Fₘₐₓ-Fₘᵢₙ) is determined. The values for 100% activity (no inhibition) and 0% activity (complete inhibition) can be obtained from control samples (samples ‘peptide only’ and ‘no peptide’). For all other samples the corresponding activity is calculated in ‘per cent’ and plotted against the Spiegelmer concentration (inhibition curve), allowing the determination of the half-maximal inhibition constant (IC50).

FIG. 3 shows the inhibition curves resulting from an experiment, that analyses the inhibitory activity of L-NOX-B 11 with full-length and truncated forms of octanoyl-ghrelin. It turns out, that the Spiegelmer inhibits the activity of all forms of octanoyl-ghrelin tested: the full-length peptide, ghrelin 1-10, and ghrelin 1-5. The IC₅₀ values show no significant deviation for all three peptides (full-length ghrelin: 7 nM, ghrelin 1-10: 9 nM, ghrelin 1-5: 5 nM). It can be concluded, that the binding region of the Spiegelmer is located at the N-terminus of ghrelin, comprising the amino acids 1-5. Binding of L-NOX-B11 to this minimal motive results in efficient inhibition of ghrelin biological activity in the cellular assay.

### EXAMPLE 4

### Discrimination of Octanoyl-Ghrelin and Desoctanoyl-Ghrelin by Ghrelin-Binding Spiegelmers

The characteristics of the binding of Spiegelmer L-NOX-B11 to ghrelin were further analysed in a competition assay, based on the method described in Example 3. In these assays, the Spiegelmer was incubated with different combinations of ghrelin peptides in the stimulation solutions prior to stimulation of cells.

The scheme of peptide combinations and the results of the experiment with full-length ghrelin are summarized in FIG. 4 (bars numbered from left to right): without any ghrelin, or with desoctanoyl-ghrelin in a final concentration of 300 nM, no stimulation of cells can be detected (bars 1 and 2), while already octanoyl-ghrelin in a concentration of 10 nM is sufficient for mediating calcium release (bar 3); further addition of 300 nM desoctanoyl-ghrelin (bar 4) does not interfere with cell stimulation, indicating that the biologically inactive desoctanoyl-ghrelin is not a receptor antagonist. The calcium release mediated by 10 nM octanoyl-ghrelin can be inhibited by a 3-fold excess of L-NOX-B11 (bar 5), and even the presence of desoctanoyl-ghrelin in a 30-fold excess (300 nM) over octanoly-ghrelin does not compete for inhibition (bar 6). In contrast, an assay concentration of 300 nM octanoyl-ghrelin and 30 nM Spiegelmer shows increased calcium release (bar 7), giving evidence that under assay conditions a stimulation enhancement with octanoyl-ghrelin can be achieved. This experiment demonstrates, that L-NOX-B11 specifically discriminates between ghrelin in the octanoyl-form and the desoctanoyl-form.

The experiment was repeated with ghrelin 1-5 instead of the full-length peptide, showing identical results (FIG. 5). However, depending on the weaker stimulatory activity of ghrelin 1-5, the signals are comparatively lower.

### EXAMPLE 5

### Requirements for Binding of L-NOX-B11 to Octanoyl-Ghrelin

The binding site for L-NOX-B11 on octanoyl-ghrelin is located at the N-terminus of the peptide (compare Example 3) and involves the octanoyl-group (compare Example 4). The importance and involvement of both components for the binding event, peptide and fatty acid group, is shown in the following experiment.

The rationale of this experiment is, that Spiegelmers bind their target peptides in an enantio-specific manner, and the octanoyl-group itself is an achiral group. If the fatty acid portion of ghrelin alone was sufficient for binding the Spiegelmer, the binding event would not be enantio-selective concerning the peptide portion; then D-NOX-B11 and L-NOX-B11 should bind D-octanoyl-ghrelin in a similar manner.

NOX-B11 was chemically synthesized as L- and D-RNA and radio-labelled using T4-Polynucleotidekinase (Invitrogen, Karlsruhe) with γ-³²[P]-ATP (Hartmann Analytic, Braunschweig). RNA was purified on a 10% denaturing polyacrylamide gel and 0, 5-5 pmol RNA were incubated with 5 μM of biotinylated D-ghrelin in binding buffer [20 mM Tris/HCl, pH 7,4; 150 mM NaCl; 5 mM KCl; 1 mM MgCl₂; 1 mM CaCl₂; 0,1% Tween-20] for 2 h at 37° C. The comparably high peptide concentration was chosen to allow monitoring of even weak Spiegelmer interactions. Subsequently, a constant amount of Streptavidin-conjugated UltraLink matrix was added. The matrix-bound ghrelin-RNA complexes were washed with binding buffer, counted in a scintillation counter (Beckman LS6500), and plotted as percentage of total binding to D-ghrelin. Each experimental group was analysed in triplicate. The results of the experiment are shown in FIG. 6.

It turned out, that the D-NOX-B11 specifically binds to D-octanoyl-ghrelin (bars 1 and 2), whereas the corresponding L-enantiomer fails (bars 3 and 4). This result indicates that the octanoyl residue mainly serves as a hydrophobic group, presenting the N-terminal GSSFL motive of the L-octanoyl-ghrelin in a conformation where the spiegelmer L-NOX-B11 efficiently binds. Both, the peptide and the octanoyl-part of L-octanoyl-ghrelin are necessary for binding L-NOX-B11.

The features of the present invention disclosed in the specification, the claims and/or the drawings may both separately and in any combination thereof be material for realizing the invention in various forms thereof.

1. A nucleic acid which binds to a bioactive ghrelin.

2. The nucleic acid according to claim 1, wherein the nucleic acid does not specifically bind to a bioactive ghrelin.

3. The nucleic acid according to claim 1, wherein the binding is expressed as a Kd value, wherein the Kd of the nucleic acid is from 10 pM to 1 μM.

4. The nucleic acid according to claim 1, wherein the bioactive ghrelin is n-octanoyl ghrelin.

5. The nucleic acid according to claim 4, wherein the n-octanoyl is attached through an ester bond to Ser at position 3 of ghrelin.

6. The nucleic acid according to claim 1, wherein the nucleic acid is an L-nucleic acid.

7. The nucleic acid according to claim 1, wherein the nucleic acid is a deoxyribonucleic acid, a ribonucleic acid or mixtures thereof.

8. The nucleic acid according to claim 1, wherein the nucleic acid comprises a secondary structure shown in FIG. 1B.

9. The nucleic acid according to claim 8, wherein the nucleic acid is variable in the internal loop structure of the secondary structure shown in FIG. 1B.

10. The nucleic acid according to claim 1, wherein the nucleic acid comprises any one of SEQ ID NOs:1-15.

11. A method for the detection of bioactive ghrelin, comprising the following steps:
(a) providing a sample which is to be tested for the presence of bioactive ghrelin,
(b) providing the nucleic acid according to claim 1, and
(c) reacting the sample with the nucleic acid,
whereby step (a) can be performed prior to step (b), or step (b) can be preformed prior to step (a).

12. The method according to claim 11, further comprising:
(d) detecting the reaction of the sample with the nucleic acid.

13. The method according to claim 11, wherein the nucleic acid of step (b) is immobilized to a surface.

14. The method according to claim 13, wherein the nucleic acid is immobilized to a surface via a covalent chemical bond between the surface and the nucleic acid or by an interaction partner of the nucleic acid.

15. The method according to claim 14, wherein the interaction partner is a nucleic acid, a polypeptide, a protein or an antibody.

16. The method according to claim 15, wherein the nucleic acid is selected from the group consisting of aptamers, spiegelmers and nucleic acids, which are at least partially complementary to the nucleic acid.

17. The method according to claim 13, wherein the nucleic acid comprises a first member of a pair of interaction partners and the surface comprises a second member of the pair of interaction partners.

18. The method according to claim 17, wherein the pair of interaction partners is selected from the group of biotin and avidin, biotin and streptavidin, and biotin and neutravidin.

19. The method according to claim 12, wherein said detecting comprises biotin, a bromo-desoxyuridine label, a digoxigenin label, a fluorescence label, a UV label, a radiolabel, or a chelator molecule.


SEQUENCE LISTING (21 sequences)

SEQ ID NO: 1 - RNA, 48 nt, Artificial
  Synthetic
cgugygnaggyanaaaacnuaarwccgaagguaaccawuccuacnacg

SEQ ID NO: 2 - RNA, 47 nt, Artificial
  Synthetic
cgugugaggcaauaaaacuuaaguccgaagguaaccaauccuacacg

SEQ ID NO: 3 - RNA, 47 nt, Artificial
  Synthetic
cgugugaggcaguaaaacuuaaguccgaagguaaccaauccuacacg

SEQ ID NO: 4 - RNA, 47 nt, Artificial
  Synthetic
cgugugaggcaauaaaacuuaaguccgaagguaaccaauccugcacg

SEQ ID NO: 5 - RNA, 47 nt, Artificial
  Synthetic
cgugugaggcaauaaaacauaaguccgaagguaaccaauccuacacg

SEQ ID NO: 6 - RNA, 47 nt, Artificial
  Synthetic
cgugugaggcaauaaaacguaaguccgaagguaaccaauccuacacg

SEQ ID NO: 7 - RNA, 49 nt, Artificial
  Synthetic
cgugugaggcaauaaaacuuguaaguccgaagguaaccaauccuacacg

SEQ ID NO: 8 - RNA, 48 nt, Artificial
  Synthetic
cgugugaggcaauaaaaacuuaaguccgaagguaaccaauccuacacg

SEQ ID NO: 9 - RNA, 50 nt, Artificial
  Synthetic
cgugcggugaggcaaaaacguaagaccgaagguaaccauuccuacccacg

SEQ ID NO: 10 - RNA, 50 nt, Artificial
  Synthetic
cgugugagguaguaaaaaaacguaaauccgaagguaaccaauccuacacg

SEQ ID NO: 11 - RNA, 53 nt, Artificial
  Synthetic
cgugugagguaguaaaaaaaaaacguaaauccgaagguaaccaauccuacacg

SEQ ID NO: 12 - RNA, 54 nt, Artificial
  Synthetic
cgugugagguaguaaaaaaaaaaacguaaauccgaagguaaccaguccuacacg

SEQ ID NO: 13 - RNA, 55 nt, Artificial
  Synthetic
cgugugagguaguaaaaaaaaaaaacguaaauccgaagguaaccaauccuacacg

SEQ ID NO: 14 - RNA, 56 nt, Artificial
  Synthetic
cgugugagguaguaaaaaaaaaaaaacguaaauccgaagguaaccaauccuacacg

SEQ ID NO: 15 - RNA, 59 nt, Artificial
  Synthetic
cgugugagguaguaaaaaaaaaaaaaaaacauaaauccgaagguaaccaauccuacacg

SEQ ID NO: 16 - protein, 28 aa, Homo sapiens
  misc_feature: human ghrelin
GSSFLSPEHQRVQQRKESKKPPAKLQPR

SEQ ID NO: 17 - protein, 10 aa, Homo sapiens
  misc_feature: amino acids 1 to 10 of human ghrelin
GSSFLSPEHQ

SEQ ID NO: 18 - protein, 5 aa, Homo sapiens
  misc_feature: amino acids 1 to 5 of human ghrelin
GSSFL

SEQ ID NO: 19 - protein, 28 aa, rat
  misc_feature: rat ghrelin
GSSFLSPEHQKAQQRKESKKPPAKLQPR

SEQ ID NO: 20 - RNA, 17 nt, Artificial
  misc_feature: 5′ flank sequence
ggagcucagacuucacu

SEQ ID NO: 21 - RNA, 18 nt, Artificial
  Synthetic
uaccacugucgguuccac